(19) United States
(12) Patent Application Publication
(10) Pub. No.: US 2004/0014063 A1
(43) Pub. Date: Jan. 22, 2004

(54) Il-6 detection for predicting risks of preterm delivery

(76) Inventors: Frederic Batteux, Paris (FR); Emmanuel Claret, Besancon (FR); Helene Trebeden, Issy-Les-Moulineaux (FR); Claudine Vermot-Desroches, Besancon (FR); Bernard Weill, Eaubonne (FR); John Wijdenes, Larnord (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/276,696

(22) PCT Filed: May 18, 2001
(86) PCT No.: PCT/FR01/01537

(30) Foreign Application Priority Data
May 18, 2000 (FR) 00/06359

(51) Int. Cl. C12Q 1/68; G01N 33/558; G01N 33/543

(52) U.S. Cl. 435/6; 436/518; 436/514

(57) ABSTRACT

The invention concerns a method for forecasting impending preterm delivery by detecting the presence or absence of IL-6 in a cervicovaginal secretion sample, in particular by detecting IL-6 mRNA by PCR or by direct detection of IL-6 through immuno-chromatography.

The invention relates to a method for diagnosing impending preterm deliveries by detecting IL6 in cervicovaginal secretions.

In the report of the present invention, “preterm delivery” defines a delivery before 34 weeks of amenorrhea; “very preterm delivery” defines a delivery before 32 weeks of amenorrhea; and “impending delivery” defines a delivery within the 14 days, preferably within the 7 days, following the diagnostic test.

Preterm deliveries occur in approximately 10% of pregnancies and are responsible for 75% to 90% of neonatal morbidity and mortality, in the absence of congenital abnormalities. Despite the progress made in the last few decades in obstetric and neonatal monitoring, the number of preterm births has remained constant or has increased.

Specifically, the diagnosis and the therapeutic treatment of a threat of preterm delivery remain unsolved clinical problems. Rapid identification of patients at risk is difficult. Conventional methods of prognosis, which are based on the analysis of obstetric history, of demographic factors and of premonitory symptoms, are neither objective, nor sensitive, nor specific. Furthermore, because the preventive treatments proposed, based on the massive use of tocolytic agents capable of delaying delivery, prove to be relatively ineffective, it is essential, in the event that a threat of preterm delivery is diagnosed, to also predict impending delivery in order to plan all the measures of neonatal intensive care to ensure the survival of the premature baby.

Many studies have been carried out, in order to search for biochemical markers correlated with a threat of preterm delivery, which would allow reliable and rapid diagnosis able to be used to monitor patients at risk.

Intrauterine infection is one of the identified causes of preterm delivery, which represents, however, only 10% to 40% of cases observed. The role of inflammation and the involvement of certain cytokines (IL8, IL1β, Il6 and TNFα) in triggering delivery, whether full term or preterm, has now been clearly established. For example, in a recent review, WINKLER et al. [J. Perinat. Med., 27, 45-60, (1999)] report that the extracellular matrix of the uterine cervix plays an essential role in maintaining the pregnancy. The rapid dilation of the cervix which precedes delivery is thought to be associated with rapid degradation of this matrix by proteases induced by a local inflammatory reaction, which is thought to be accompanied by extravasation of neutrophils and of leukocytes and also by an increase in the concentration of certain cytokines (IL8, IL1β, Il6 and TNFα).

It has been proposed to assay these cytokines, and also inflammation proteins (fibronectin), in the amniotic fluid in order to diagnose the risk of preterm and/or impending delivery. For example, HILLIER et al. [Obstetrics & Gynecology, 6, 941-948, (1993)] report that high values for IL6 in the amniotic fluid allow an impending (<7 days) delivery to be diagnosed with an 85% sensitivity.

However, amniocentesis is an invasive and potentially risky technique poorly suited to medical monitoring of patients at risk, which requires regular tests (every week) to be carried out. Studies have therefore been carried out to determine whether these markers are also present in cervicovaginal secretions, and whether assaying them would make it possible to assess the risk of preterm and/or impending delivery.

The results reported are divergent. In the case of fibronectin, INGLIS et al. [Am. J. Obst Gynecol., 171, 5-10, (1994)] studied the immunoenzymatic assaying of total fibronectin and/or of fetal fibronectin, in vaginal secretions. The results obtained show that assaying total fibronectin makes it possible to forecast the occurrence of a delivery within 2 to 3 weeks (too long a period to be of advantage to clinicians), and that it is not specific for a preterm delivery. As regards fetal fibronectin, which is normally present only in the amniotic fluid and the placenta, assaying it in cervicovaginal secretions can be used only in cases of a threat of preterm delivery in which the membranes have been ruptured or damaged.

In the case of IL8, TANAKA et al. [Am. J. Obstet. Gynecol., 179, 644-649, (1998)] consider that an increase therein, and also in ILl, in cervicovaginal secretions is associated with a risk of preterm delivery. However, it has also been reported that considerable amounts of IL-8 in cervicovaginal secretions make it possible to diagnose an intrauterine infection with high sensitivity (80%) [RIZZO-et al., J. Perinat. Med., 25, 461-468, (1997); RIZZO et al., Ultrasound Obstet. Gynecol., 12, 86-92, (1998)], but do not appear to constitute a reliable marker for predicting a preterm delivery [RIZZO et al., 1997, cited above].

As regards IL6, several studies [LOCKWOOD et al., Am. J. Obst. Gynecol., 171, 1097-1102, (1994); INGLIS et al., Am. J. Obst. Gynecol., 171, 5-10, (1994)]; are in agreement in concluding that there is an increase in the amount of IL6 in cervicovaginal secretions in the case of preterm delivery, but diverge with regard to the threshold value to be selected. RIZZO et al. [Am. J. Obstet. Gynecol., 175, 812-817, (1996)] consider, moreover, that the amount of IL6 in cervicovaginal secretions constitute a marker for infection of the amniotic fluid. No link has been established between the amount of IL6 in cervicovaginal secretions and impending delivery. The interpretation of the results is, in any case, made difficult by the fact that varying and sometimes high amounts of IL6 have been detected in samples taken from control patients.

The inventors have undertaken to study the local activation of cellular immunity in patients, compared with control patients having a normal pregnancy with no risk of a threat of preterm delivery, in order in particular to determine whether specific cytokines present in cervicovaginal secretions are produced locally. Specifically, vaginal secretions are composed: of a solid phase containing exfoliated epithelial cells and leukocytes, and also polymerized glycoproteins secreted into the cervical mucus and bacteria, and of a liquid phase containing electrolytes and also transudated or secreted proteins (immunoglobulins, glycoproteins (cytokines), albumin and lactoferrin) [BELEC et al., Clinical and Diagnostic Laboratory Immunology, 2, 57-61, (1995)].

With this aim, the inventors, by RT-PCR, have looked for the presence of mRNAs of the cytokines IL6, IL8, IL10 and IL13 in cell pellets obtained from the cervicovaginal secretions of the 2 groups of patients. They thus noted that IL8 and IL10 mRNAs were detectable both in the controls (33.3% and 16.7%) and in the patients at risk from a threat of preterm delivery (40.3% and 48.1%), but that, on the other hand, IL6 and IL13 mRNAs were present in, respectively, 10.9% and 6.2% of patients, but were not detected in the patients of the control group. The inventors also noted that IL6 expression in the cervicovaginal secretions was a marker which correlated with prematurity (delivery before the 34th week of gestation p=0.02) and extreme prematurity (delivery before the 32nd week of gestation p=0.049), and made it possible to diagnose an impending preterm delivery within a period of less than 14 days (p=0.001), or even within a period of less than 7 days (p=0.036). On the other hand, IL8 correlates with maternal infection and neonatal infection, but it does not correlate with an impending delivery.

As these results, and in particular the absence of IL6 in patients of the control group, were not in agreement with the observations of the prior art, which report the presence of IL6 in cervicovaginal secretions, including in the case of patients having a normal pregnancy, the inventors investigated the reasons for this disagreement. They supposed that it could be due to the use, in the prior art, of immunoenzymatic assaying methods of the ELISA type. Specifically, vaginal secretions constitute a complex, relatively nonhomogeneous medium in which the “background noise” which ensues in particular from the turbidity of the sample may interfere with ELISA assays.

The inventors therefore chose to look for IL6 in cervicovaginal secretions using methods of immuno-detection on a solid support, which, unlike ELISA techniques, do not suffer from interference due to problems of background noise. The results obtained confirm the absence of IL6 in the cervicovaginal secretions of the patients showing no threat of preterm delivery, and the correlation between the presence of IL6 and impending delivery.

The inventors also observe that IL6 was detected in the cervicovaginal secretions even in the absence of rupturing of the placental membranes, causing IL6 from the amniotic fluid to pass into the cervicovaginal secretions. They therefore noted that, surprisingly, there was a local production of IL6 by the leukocytes of the mucosa of the vagina and of the uterine cervix, correlated with a threat of impending preterm delivery

These results make it possible to propose a novel method for predicting risks of preterm delivery, and in particular an impending preterm delivery.

A subject of the present invention is a method for forecasting impending preterm delivery, characterized in that it comprises detecting the presence or absence of IL6 expression in a cervicovaginal secretion sample obtained from a patient to be tested. Conventionally, cervicovaginal secretions are taken at the ectocervix and at the posterior formix.

In accordance with the invention, detection of the presence or absence of IL6 in cervicovaginal secretions can in particular be carried out:

by detecting IL6 mRNA or

by directly detecting IL6.

The detection of IL6 mRNA can be carried out by conventional molecular biology methods, with oligo-nucleotides specific for the sequence encoding IL6, for example using the mRNA extracted from a cell pellet obtained by centrifugation of cervicovaginal secretions. Advantageously, a detection method comprising specific amplification, after reverse transcription, of IL6 mRNA will be chosen.

The direct detection of IL6 can be carried out in particular by immunodetection, preferably using a supernatant from centrifugation of cervicovaginal secretions. The immunoenzymatic assaying techniques of the ELISA type described in the prior art do not permit reliable detection, for the reasons of background noise mentioned above.

A technique of detection on a solid support by immuno-chromatography will therefore be chosen, the general principle which, known in itself, is as follows:

the sample liable to contain the analyte being sought is brought into contact with a first antibody (revealing antibody), which is directed against said analyte and is labeled (generally using colored microparticles). The mixture thus formed is deposited onto a chromatography support to which a second antibody (capture antibody), also directed against said analyte, is attached. If the analyte is present in the sample, the analyte/labeled revealing antibody complex is detected at the place where its migration is stopped by formation of a second complex with the capture antibody.

To implement the present invention, two anti-IL6 antibodies which recognize different epitopes of the IL6 molecule will be chosen as revealing antibody and capture antibody, respectively. Antibodies which are most particularly suitable are chosen from the monoclonal antibodies BE-4 (C.N.C.M I-911), BF-6 (C.N.C.M I-912) and BE-8 (C.N.C.M I-913) described in application EP 0 430 193 in the name of the CENTRE NATIONAL DE TRANSFUSION SANGUINE [National Center for Blood Transfusion] of Besancon and of BIOTEST PHARMA GMBH. Advantageously, the BE-8 antibody will be chosen as capture antibody and the BE-4 antibody will be chosen as revealing antibody.

The labeling of the revealing antibody and the attaching of the capture antibody to the chromatography support are done according to methods known, in themselves, to those skilled in the art.

The revealing antibody may, for example, be labeled with microparticles of colored gelatin, liposomes, microparticles of metal, in particular of colloidal gold etc.

In the context of the present invention, various variants and various improvements of the abovementioned detection by immunochromatography may be used. For example, it is possible to use ready-to-use devices for detection by immunochromatography, which are commercially available and can be combined with antibodies chosen for detecting a given analyte. Generally, these devices are in the form of small strips comprising a chromatography support (for example a nitrocellulose membrane) to which the capture antibody can be attached, and a depositing area intended to receive the sample to be analyzed; between the area of attachment of the capture antibody and the depositing area, and adjacent to the latter, there is an area intended to receive the labeled revealing antibody. After attachment of the capture antibody to the chromatography support, and impregnation with the revealing antibody of the area intended to receive it, the device is ready to be used for the analysis. This is carried out simply by depositing the sample to be analyzed onto the area provided for this purpose; the possible formation of the analyte/revealing antibody complex occurs when the sample migrates through the area impregnated with this revealing antibody; this complex is then detected visually, after rinsing the chromatography support, in the form of a colored band where the capture antibody was attached. The detection can thus be carried out in a few minutes.

The detection of IL6 by immunochromatography, according to the methods defined above, constitutes an embodiment of the invention which is particularly suitable for monitoring patients at risk, since it does not require any bulky and expensive equipment, can be easily carried out by the physician or the medical personnel at the patient's bedside, and makes it possible to obtain a response rapidly and thus to take the necessary steps in the event of impending delivery being forecasted.

Besides the arrangements above, the invention also comprises other arrangements which will emerge from the following description, which refers to examples of implementation of the method which is the subject of the present invention. It should be clearly understood, however, that these examples are given only by way of illustration of the subject of the invention, of which they in no way constitute a limitation.

### EXAMPLE 1

### Expression of Inflammatory Cytokine mRNAS in the Cervicovaginal Secretions of Patients with a Threat of Preterm Delivery

A-Materials and Methods

1-Description of the Population Studied

The population studied consists of 307 patients hospitalized at the Maternite Port Royal [Port Royal Maternity Hospital] (Paris), admitted for threat of preterm delivery between 13 and 35 weeks of amenorrhea (WA). The control group is composed of thirty pregnant females (more than one trimester) with no threat of preterm delivery.

The inclusion criteria are defined according to the clinical and echographic examination.

Among these patients, 258 (84%) have intact membranes.

2-Obstetric Characteristics of the Population Studied

These patients were monitored up to the delivery in order to determine:

the period of time between the sampling day and the delivery date,

the prematurity (<32 weeks of amenorrhea (extreme prematurity) or <34 weeks of amenorrhea (prematurity)).

In the population studied, 30.9% of the patients delivered prematurely (<34WA) and 10.7% delivered within the seven days after admission. 18.9% delivered within the fourteen days following admission.

3-Cervicovaginal Sampling

The cervicovaginal secretions were collected by aspiration at the ectocervix and at the posterior fornix using a sterile plastic sampling pipette with a foam tip. The macroscopically hemorrhagic samples were excluded at this step. The cells were centrifuged at 1 500 rpm/5 min and washed twice in PBS buffer. The epithelial cells and the leukocytes were counted on Malassez cells. The cell pellet was then resuspended in 500 μl of TRI-REAGENT (EUROMEDEX, Souffelmeyersheim, France), for the purpose of extracting the total RNA.

4-Extraction of the total RNA and Reverse Transcription

The total RNA of vaginal secretion cells was extracted by the guanidine isothiocyanate-phenol technique, and then treated with DNAse I (BOEHRINGER MANHEIM, Meylan, France) in order to remove all traces of genomic DNA. 3 μg of RNA were transcribed to cDNA for two hours at 42° C., in the presence of 6 IU of reverse transcriptase (PROMEGA, Charbonniéres, France), in a final volume of 40 μl.

5-PCR

100 ng of cDNA were then amplified by PCR, in the presence of 2 IU of Taq DNA polymerase (ATGC, Noisy le Grand) and oligonucleotides which were specific: for human actin, for IL6, for IL8, for IL10 or for IL13. After an initial denaturation of 5 min at 94° C. and 3 min of hybridation at 57° C., 35 amplification cycles were performed under the following conditions (elongation: 30 sec at 72° C., denaturation: 30 sec at 94° C. and hybridization: 30 sec at 57° C.), followed by an elongation of 7 min at 72° C., and the products were maintained at 4° C.

For the positive samples, the messenger RNAs are then quantified by competitive PCR, using a known number of copies of the competing plasma (PQA1 for IL6 or PQB2 for actin). After separation of the products by 2% agarose gel electrophoresis in the presence of ethidium bromide, the gel is photographed and the intensity of the bands is measured using a densitometer (Vilbert-Loumat, Marne la Vallée, France).

6-Search for an Infection in the Mother and the Child

Infection in the mother is assessed on the basis of temperature, the cytobacteriological examination of the urine, serum CRP, the complete blood count (leuko-cytosis) and the vaginal sample. Neonatal infection is assessed on the basis of fever, serum CRP, the complete blood count and the samples taken at the orifices for bacteriological study.

7-Assaying of Inflammation Parameters (CRP and Total Fibronectin)

The serum CRP is assayed by nephelometry and the fetal fibronectin is quantified in the cervicovaginal secretions using conventional immunoenzymatic techniques (ELISA), with commercial kits.

8-Statistical Analysis

Association of cervicovaginal secretion cytokines with prematurity and with the period of time between the sampling day and the delivery date was performed by multiple regression. The differences between the groups were determined using the χ² test and Fischer's exact test.

B-Results

1-Demonstration of Markers Specific for a Threat of Preterm Delivery

Production of the cytokines IL6, IL8, IL10 and IL13 was analyzed by RT-PCR in the vaginal secretions of 129 patients at risk of a threat of preterm delivery, and of 30 control patients who were pregnant with no risk of a threat of preterm delivery. The presence or absence of these cytokines in the samples of the 2 groups was determined and the results obtained, expressed as percentages, are given in table I below.

TABLE I

| | Threat of preterm | |
|---|---|---|
| | delivery | Control |
| IL6 | 10.9 | 0 |
| IL8 | 40.3 | 33.3 |
| IL10 | 48.1 | 16.7 |
| IL13 | 6.2 | 0 |

These results show that IL6 and IL13 are cytokines specific for a threat of preterm delivery, which can potentially be used for diagnosis.

Since the signal observed by RT-PCR with IL13 was much weaker than that observed with IL6, IL6 was therefore selected as a more sensitive and therefore a potentially more advantageous marker for diagnostic application. The number of IL6 messengers was determined by quantitative PCR; an average number of 230 000 copies per positive sample observed, which corresponds to 100 to 1 000 times more copies than for actin.

2-Association of the Various Cytokines with Preterm Delivery

The results are given in table II below; the significant associations are indicated in bold with their significance threshold.

TABLE II

| Cytokines | <32 WA | 32 WA < < 34 WA | >34 WA |
|---|---|---|---|
| IL6 | 31.1% (p = 0.001) | 26.3% (p = 0.001) | 4.25% |
| TL8 | 75.4% (p = 0.004) | 64.2% | 56.6% |
| IL10 | 73.7% (p = 0.029) | 48.7% | 60.4% |
| IL13 | 14.75% | 18.9% | 17.9% |

The results show a significant association between the presence of IL6, IL8 and IL10 in cervicovaginal secretions and extreme prematurity (<32 WA). On the other hand, only the presence of IL6 is associated both with extreme prematurity (<32 WA) and with prematurity (<34 WA).

3-Association of Cytokines with Delivery within the 7 Days or the 14 Days after Admission

The results are given in table III below; the significant associations are indicated in bold with their significance threshold.

TABLE III

| Cytokine | <7 d | 7 d < < 14 d | >14 d |
|---|---|---|---|
| IL6 | 33.3% (p = 0.001) | 39.7% (p = 0.001) | 4.4% |
| IL8 | 81.2% (p = 0.005) | 75.8% (p = 0.004) | 55% |
| IL10 | 72.7% | 68.9% | 59.8% |
| IL13 | 21.2% | 18.97% | 18.1% |

The results show a significant association between the presence of 1L6 in cervicovaginal secretions and delivery within the 7 days or the 14 days following admission.

In addition, in the population of 258 patients having intact membranes, the presence of IL6 in the cervicovaginal secretions is significantly associated with a delivery time of less than 14 days (p=0.003).

All of the results demonstrate that the detection of IL6 mRNA by RT-PCR, in the cervicovaginal secretions of patients at risk from a threat of preterm delivery therefore represents a reliable method for forecasting an impending preterm delivery [<7 days (p=0.001); <14 days (p=0.001)].

### EXAMPLE 2

### Detection of IL6 by Immunochromatography For Diagnosing a Preterm Delivery

A-Materials and Methods

1-Preparation of the Immunochromatography Device

An immunochromatography device marketed by GELMAN SCIENCES was used. The chromatography support consists of a membrane made of polyethersulfone, which is more resistant and gives greater sensitivity of detection than the nitrocellulose conventionally used.

Reagents list

membrane (PREDATOR, PALL Gelman Sciences)

Glass wool (PALL Gelman Sciences)

Absorbent paper (PALL Gelman Sciences)

Revealing antibody (BE-4, C.N.C.M. I-911)

Capture antibody (BE-8, C.N.C.M. I-913)

Goat anti-mouse IgG antibody (control)

Sodium citrate

Borax buffer (20 mM or 2 mM Na₂B₄O₇.2H₂O, pH 9.3)

BSA

Tween 20

2-Preparation of the Revealing Antibody (Antibody Coupled to Colloidal Gold)

A-Preparation of Colloidal Gold

The solution is prepared in a 500 ml Erlenmeyer flask. 4 ml of a 1% gold chloride solution are added to 200 ml of boiling distilled water. 12 ml of a 1% sodium citrate solution are then added with vigorous stirring. The solution obtained is again heated to boiling until a color resembling that of wine is obtained (approximately 3 minutes). The solution is then stored at ambient temperature in the dark.

B-Preparation of the Antibody to be Coupled to the Colloidal Gold (Revealing Antibody)

The BE-4 antibody is dialyzed overnight against distilled water, and then its protein content is assayed and it is stored at −80° C.

C-Coupling of the Antibody to the Colloidal Gold

25 μg of BE-4 antibody, and then 100 μl of 20 mM BORAX buffer, are added to 1 ml of colloidal gold. The solution obtained is incubated for 1 h at ambient temperature. 100 μl of 20 mM BORAX buffer containing 1% BSA are added and the solution is centrifuged for 15 min at 1 500 rpm. After removal of the supernatant, the pellet is resuspended in 1 ml of 2 mM BORAX buffer containing 0.1% of BSA and the solution is centrifuged for 50 min at 1 500 rpm. After removal of the supernatant, the pellet is taken up in 250 μl of 2 mM BORAX buffer containing 0.1% of BSA. The antibody conjugated to colloidal gold is stored at 4° C., in the dark.

3-Assembly of the Immunochromatography Device

A-Preparation of the Glass Wool Containing the Revealing Antibody

250 μl of the BE-4 antibody coupled to colloidal gold are added to 1 ml of 100 mM Tris HCl buffer, pH 8.0; 0.5% BSA; 0.1% Tween 20 and 5% sucrose.

Rectangles of glass wool (L:20 mm×W:5 mm) are immersed in the solution of antibody coupled to colloidal gold and then dried in an oven at 58° C. for 30 min, and stored under vacuum, in a dry atmosphere, at ambient temperature.

B-Assembly of The Device

The 2 rectangles of absorbent paper are cut out (L:16 mm×W:4 mm and L:18 mm×W:4 mm).

A small strip of membrane is cut out (L:59 mm×W:25 mm), 8 μl of capture antibody (BE-8) and of control antibody (anti-mouse IgG), at 1 mg/ml, are deposited onto a line perpendicular to the membrane, and then the membrane is dried in a desiccator. Small strips 4 mm in width are cut out and the various elements of the immunochromatography device are assembled according to the manufacturer's instructions.

4-Detection of IL6 in Cervicovaginal Secretions by Immunochromatography

The population studied, the clinical parameters analyzed and the methods for taking the cervicovaginal secretion samples are described in example 1 above.

The secretions are diluted in 300 μl of PBS buffer, pH 7.4, and the mixture is stirred and then centrifuged for 5 min at 2 500 rpm. 200 μl of the supernatant are deposited onto the area of the immunochromatography device provided for this purpose.

The migration is allowed to take place for 60 min; after rinsing of the membrane, the appearance of a brown band at the site where the capture antibody was attached reveals the presence of IL6 in the sample.

B-Results

The presence of IL6 was analyzed on 41 samples from patients with a threat of preterm delivery, included in the study of example 1. Among these patients, 37 have intact membranes and 4 exhibit a rupture of the membranes.

Four samples from patients exhibiting a rupture of the membranes give negative results by RT-PCR and positive results by immunochromatography.

These results show a 95% correlation between the immunochromatography and the RT-PCR.

In the absence of rupture of the membranes, the immunochromatography is as sensitive as the RT-PCR, for detecting IL6 in the cervicovaginal secretions.

Consequently, the method for detecting IL6 in cervicovaginal secretions by immunochromatography, of the invention, is as sensitive as the method for detecting IL6 mRNA by RT-PCR.

1. A method for forecasting impending preterm delivery, characterized in that it comprises detecting the presence or absence of IL6 in a cervicovaginal secretion sample obtained from a patient to be tested.

2. The method as claimed in claim 1, characterized in that IL6 mRNA is detected.

3. The method as claimed in claim 1, characterized in that IL6 is directly detected.

4. The method as claimed in claim 3, characterized in that the IL6 is detected by immunochromatography.

5. The method as claimed in claim 4, characterized in that it uses at least two anti-IL6 monoclonal antibodies chosen from: BE-4 (C.N.C.M I-911), BF-6 (C.N.C.M I-912) and BE-8 (C.N.C.M I-913).

6. The method as claimed in claim 5, characterized in that the capture antibody is the BE-8 antibody.

7. The method as claimed in claim 5, characterized in that the revealing antibody is the BE-4 antibody.

8. The use of a device for detection by immunochromatography, for detecting IL6 in cervicovaginal secretions for the purpose of forecasting impending preterm delivery.
